(19) United States
(12) Patent Application Publication
(10) Pub. No.: US 2010/0217126 A1
(43) Pub. Date: Aug. 26, 2010

(54) ULTRASONIC DIAGNOSTIC APPARATUS, METHOD OF DISPLAYING ULTRASONIC IMAGES AND PROGRAM

(76) Inventors: Tsutomu Yawata, Tokyo (JP); Yasuyo Saito, Tokyo (JP)

Correspondence Address: PATRICK W. RASCHE (20459);ARMSTRONG TEASDALE LLP, 7700 Forsyth Boulevard, Suite 1800, St. Louis, MO, 63105, US

(21) Appl. No.: 12/712,928

(22) Filed: Feb. 25, 2010

(30) Foreign Application Priority Data
Feb. 25, 2009 (JP) 2009-042089

(51) Int. Cl. A61B 8/14

(52) U.S. Cl. 600/443

(57) ABSTRACT

An ultrasonic diagnostic apparatus includes a storage unit configured to store echo data obtained by transmitting and receiving an ultrasonic wave to and from a subject, an image processing unit configured to perform image processing with different image processing parameters with respect to the echo data stored in the storage unit, and a display processing unit configured to generate a plurality of ultrasonic images based on the processed echo data and to display the plurality of ultrasonic images.

### CROSS REFERENCE TO RELATED APPLICATIONS

This application claims the benefit of Japanese Patent Application No. 2009-042089 filed Feb. 25, 2009, which is hereby incorporated by reference in its entirety.

### BACKGROUND OF THE INVENTION

The embodiments described herein relate to an ultrasonic diagnostic apparatus and program.

An ultrasonic diagnostic apparatus carries out a scan on a subject with an ultrasonic probe to acquire echo data. This echo data is subjected to image processing using various image processing parameters and then scan conversion is carried out to generate an ultrasonic image, and this image is displayed on a display unit.

Examples of the above image processing parameters include gain, dynamic range, TGC (Time Gain Control), and the like. The brightness of an ultrasonic image is adjusted by adjusting gain and the resolution of an ultrasonic image is adjusted by adjusting dynamic range. The adjustment of TGC makes it possible to obtain an ultrasonic image even in brightness in the direction of depth. An operator sets image processing parameters deemed optimum so that an ultrasonic image of image quality suitable for diagnosis can be obtained.

Setting of image processing parameters has been conventionally made by an operator by varying each image processing parameter at an operating portion while viewing one ultrasonic image displayed on a display unit. However, even when the operator finds some image processing parameters deemed optimum while he/she is adjusting the image processing parameters, it used to be impossible to compare ultrasonic images generated with different image processing parameters. Therefore, it has been difficult to make setting in a short time and what is worse it has been sometimes impossible to set optimum image processing parameters. To cope with this, Japanese Unexamined Patent Publication No. Hei 8 (1996)-266539 proposes an ultrasonic diagnostic apparatus in which different numeric values are set for various image processing parameters when image processing is carried out and multiple obtained ultrasonic images are displayed on a display unit in alignment.

In the ultrasonic diagnostic apparatus disclosed in Japanese Unexamined Patent Publication No. Hei 8 (1996)-266539, the multiple ultrasonic images displayed on a display unit in alignment are real-time images or images frozen during a scan and are not images obtained after the end of a scan. There are cases where ultrasonic images stored in the apparatus by an inexperienced operator setting image processing parameters and carrying out a san are viewed by an experienced operator after the end of the scan. In these cases, the experienced operator may desire to re-set an image processing parameter when he/she views the multiple ultrasonic images displayed in alignment.

### BRIEF DESCRIPTION OF THE INVENTION

A first aspect of the invention an ultrasonic diagnostic apparatus characterized in that includes: a storage unit that stores echo data obtained by transmitting and receiving an ultrasonic wave to and from a subject; an image processing unit that carries out image processing on the echo data stored in this storage unit with different image processing parameters; and a display processing unit that generates multiple ultrasonic images based on data that underwent image processing at this image processing unit and displays these images in alignment.

A second aspect of the invention is an ultrasonic diagnostic apparatus characterized in that the above multiple ultrasonic images are either moving images or still images in the first aspect of the invention.

A third aspect of the invention is an ultrasonic diagnostic apparatus characterized in that the following is implemented in the first or second aspect of the invention: with respect to frame data of an identical time phase comprised of echo data obtained by transmitting and receiving an ultrasonic wave to and from a subject, the following is implemented: the image processing unit carries out image processing with different image processing parameters; and the display processing unit generates and displays the multiple ultrasonic images.

A fourth aspect of the invention is an ultrasonic diagnostic apparatus characterized in that the following is implemented in the third aspect of the invention: with respect to unit data comprised of multiple pieces of frame data, the above image processing unit carries out image processing on each piece of frame data with different image processing parameters; and the above display processing unit generates the multiple ultrasonic images with respect to each piece of frame data and displays moving images.

A fifth aspect of the invention is an ultrasonic diagnostic apparatus characterized in that the following is implemented in the first or second aspect of the invention: with respect to frame data of different time phases, comprised of echo data obtained by transmitting and receiving an ultrasonic wave to and from a subject, the above image processing unit carries out image processing with different image processing parameters; and the above display processing unit generates and displays the multiple ultrasonic images.

A sixth aspect of the invention is an ultrasonic diagnostic apparatus characterized in that the following is implemented in the fifth aspect of the invention: with respect to each piece of divided unit data obtained by dividing unit data comprised of multiple pieces of frame data into a predetermined number of frames, the following is implemented: the above image processing unit carries out image processing on each piece of frame data with different image processing parameters; and the above display processing unit generates the multiple ultrasonic images with respect to each piece of frame data and displays moving images.

A seventh aspect of the invention is an ultrasonic diagnostic apparatus characterized in that the following is implemented in the sixth aspect of the invention: with respect to at least any one of the above pieces of divided unit data, the above image processing unit carries out image processing with different image processing parameters; and the above display processing unit generates and displays the multiple ultrasonic images.

An eighth aspect of the invention is an ultrasonic diagnostic apparatus characterized in that in any of the first to seventh aspects of the invention, the above image processing unit carries out image processing with different image processing parameters in parallel.

A ninth aspect of the invention is an ultrasonic diagnostic apparatus characterized in that in any of the first to eighth aspects of the invention, the image processing parameters are gain, dynamic range, and sensitivity time gain.

A tenth aspect of the invention is an ultrasonic image display control program characterized in that it causes a computer to carry out the following functions: an image processing function of reading echo data, obtained by transmitting and receiving an ultrasonic wave to and from a subject, from a storage unit and carrying out image processing with different image processing parameters; and a display processing function of generating multiple ultrasonic images based on data that underwent image processing by this image processing function and displaying the images in alignment.

According to embodiments of the invention, echo data that has not undergone image processing yet is stored in the above storage unit. Even after the end of a scan, therefore, it is possible to carry out image processing on the echo data with different image processing parameters and to generate multiple ultrasonic images based on data that underwent this image processing and display the images in alignment. At this time, the echo data stored in the storage unit is data that has not undergone image processing. Therefore, it is possible to set any image processing parameter to carry out image processing and display multiple ultrasonic images.

Further objects and advantages of the present invention will be apparent from the following description of the preferred embodiments of the invention as illustrated in the accompanying drawings.

### BRIEF DESCRIPTION OF THE DRAWINGS

FIG. 1 is a block diagram illustrating the general configuration of an example of an ultrasonic diagnostic apparatus in an embodiment of the invention.

FIG. 2 is a block diagram illustrating the configuration of the display control unit in the ultrasonic diagnostic apparatus illustrated in FIG. 1.

FIG. 3 is a diagram illustrating ultrasonic images displayed on a display unit.

FIG. 4 is an explanatory diagram of unit data comprised of multiple pieces of frame data.

FIG. 5 is an explanatory diagram of divided unit data obtained by dividing unit data comprised of multiple pieces of frame data.

### DETAILED DESCRIPTION OF THE INVENTION

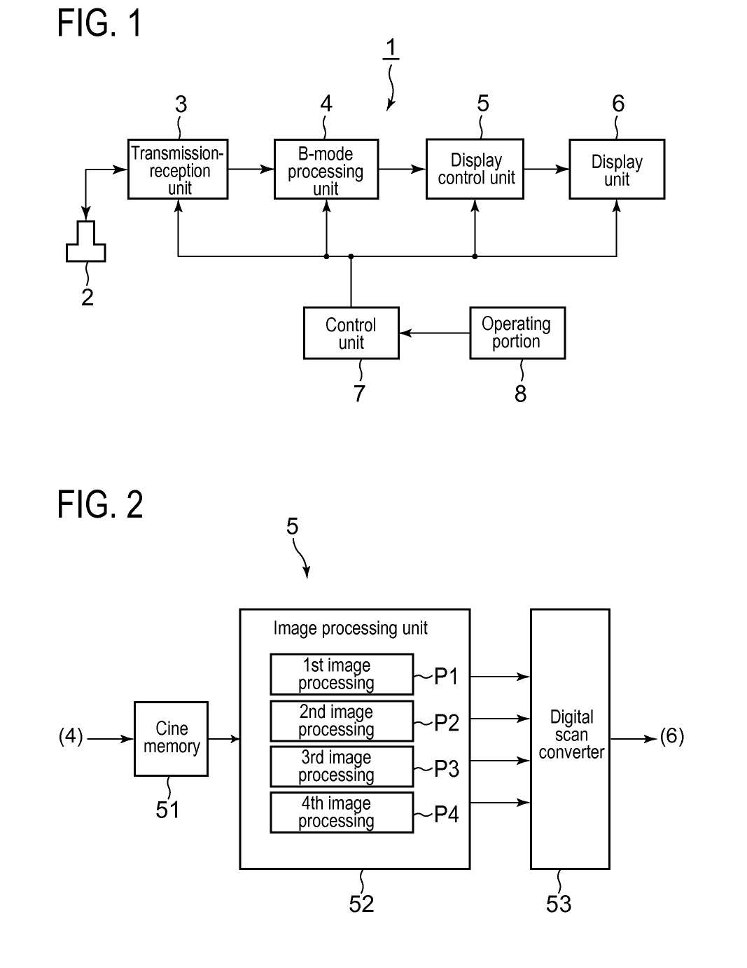

Hereafter, detailed description will be given to an embodiment of the invention with reference to the drawings. As illustrated in FIG. 1, the ultrasonic diagnostic apparatus 1 in this example includes: an ultrasonic probe 2 that transmits and receives ultrasonic waves; a transmission-reception unit 3 that drives the ultrasonic probe 2 to scan a scan surface and carries out signal processing, such as phase rectifying addition, on an echo signal obtained through the ultrasonic probe 2 to obtain echo data with respect to each sound ray; a B-mode processing unit 4 that carries out logarithmic compression, envelop detection, and the like on echo data outputted from the transmission-reception unit 3; a display control unit 5 that generates ultrasonic images (B-mode images) to be displayed on a display unit 6, described later, based on echo data from the B-mode processing unit 4 and causes the display unit 6 to display the images; and the display unit 6 on which ultrasonic images are displayed.

The ultrasonic diagnostic apparatus 1 further includes: a control unit 7 that controls the entire apparatus; and an operating portion 8, including a keyboard, a pointing device, and the like, through which an operator inputs an instruction. The control unit 7 is comprised of CPU (Central Processing Unit) and executes a control program stored in a storage device, not shown, to carry out the functions of each part of the ultrasonic diagnostic apparatus 1. Examples of these functions include the image processing function and the display processing function, described later, of the display control unit 5.

As illustrated in FIG. 2, the display control unit 5 includes a cine memory 51, an image processing unit 52, and a digital scan converter (DSC) 53. In the cine memory 51, echo data outputted from the B-mode processing unit 4 is stored. The echo data cited here refers to data that is obtained by transmitting and receiving an ultrasonic wave by the ultrasonic probe 2 and has not undergone image processing at the image processing unit 52. That is, the echo data refers to raw data. Since raw data is stored in the cine memory 51 as mentioned above, it is possible to set any image processing parameter to carry out image processing even after the end of a scan. The cine memory 51 is an example of embodiments of the storage unit in the invention.

The image processing unit 52 reads echo data stored in the cine memory 51 and carries out image processing with different image processing parameters (image processing function). The control unit 7 reads a program stored in the storage device (not shown) and causes the image processing unit 52 to carry out the above image processing function. The image processing unit 52 is an example of embodiments of the image processing unit in the invention.

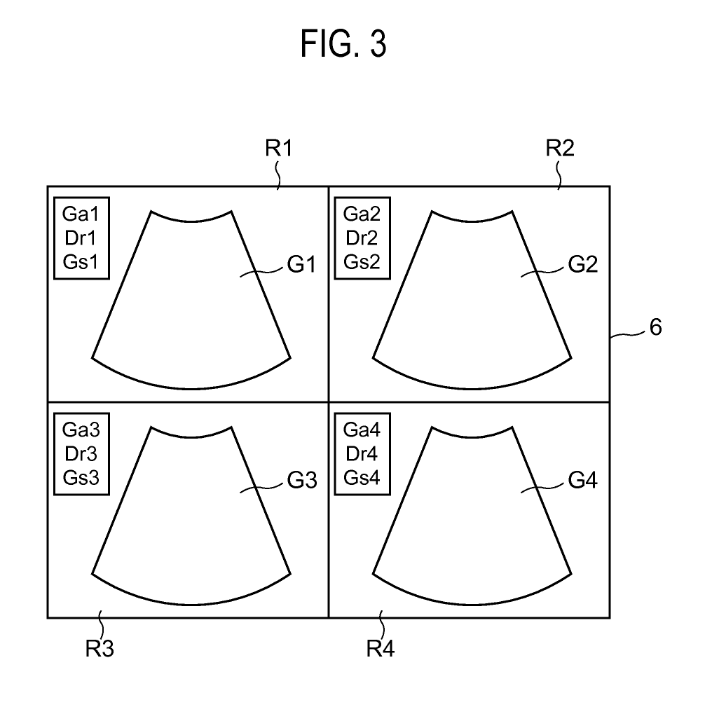

Concrete description will be given to the image processing function carried out by the image processing unit 52. In this example, the image processing unit 52 carries out first image processing P1, second image processing P2, third image processing P3, and fourth image processing P4 in parallel with image processing parameters having four different values. In each series of image processing P1 to P4, multiple series of processing are carried out. Examples of various image processing parameters set in these series of processing include gain, dynamic range, sensitivity time gain, and the like. Therefore, different numeric values are set for gain, dynamic range, sensitivity time gain, and the like for the respective series of image processing P1 to P4. Specifically, as shown in FIG. 3, gain Ga1, dynamic range Dr1, sensitivity time gain Gs1, and the like are set for the first image processing P1; gain Ga2, dynamic range Dr2, sensitivity time gain Gs2, and the like are set for the second image processing P2; gain Ga3, dynamic range Dr3, sensitivity time gain Gs3, and the like are set for the third image processing P3; and gain Ga4, dynamic range Dr4, sensitivity time gain Gs4, and the like are set for the fourth image processing P4.

These various image processing parameters are inputted at the operating portion 8 and stored in the storage device through the control unit 7. The control unit 7 reads the various image processing parameters stored in the storage device and causes the image processing unit 52 to carry out image processing.

The digital scan converter 53 subjects data obtained through each series of the image processing P1 to P4 by the image processing unit 52 to scan conversion. It thereby generates first ultrasonic image G1, second ultrasonic image G2, third ultrasonic image G3, and fourth ultrasonic image G4 and displays these images on the display unit 6 in alignment as illustrated in FIG. 3 (display processing function). The control unit 7 reads a program stored in the storage device and causes the digital scan converter 53 to carry out the display processing function. The digital scan converter 53 is an example of embodiments of the display processing unit in the invention.

Concrete description will be given to display by the display unit 6. This display unit 6 is divided into four regions R1, R2, R3, R4 and the ultrasonic images G1 to G4 are respectively displayed in the individual regions R1 to R4. The ultrasonic image G1 displayed in the region R1 is an image generated based on data obtained by the first image processing P1; and the ultrasonic image G2 displayed in the region R2 is an image generated based on data obtained by the second image processing P2. The ultrasonic image G3 displayed in the region R3 is an image generated based on data obtained by the third image processing P3; and the ultrasonic image G4 displayed in the region R4 is an image generated based on data obtained by the fourth image processing P4.

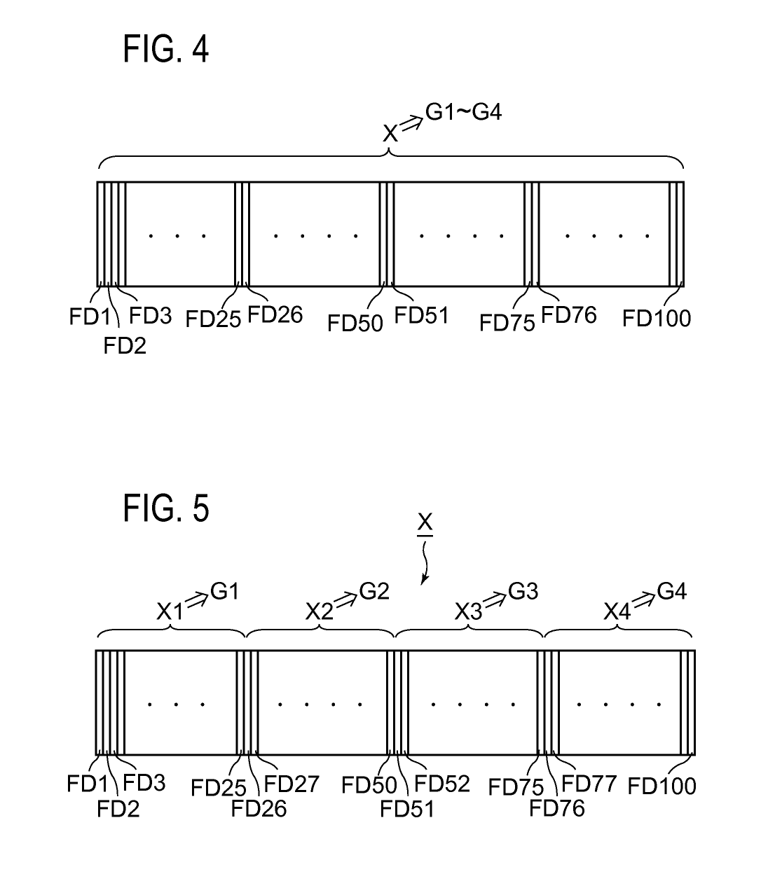

Description will be given to the operation of the ultrasonic diagnostic apparatus 1 in this example. When a ultrasonic wave is transmitted and received to and from a subject by the ultrasonic probe 2, processing is carried out at the transmission-reception unit 3 and the B-mode processing unit 4 and obtained echo data is stored in the cine memory 51. The echo data stored in the cine memory 51 is data with respect to each sound ray and an aggregate of the echo data equivalent to one frame comprises frame data FD. In the cine memory 51, there are stored 100 pieces of frame data FD (only part thereof is shown in FIG. 4), frame data FD1 to FD100, acquired over time, as illustrated in FIG. 4.

When operation for displaying multiple ultrasonic images generated with different image processing parameters is performed at the operating portion 8, the image processing unit 52 carries out the following processing: it sequentially reads echo data with respect to each sound ray stored in the cine memory 51 and carries out the individual series of image processing P1 to P4. The image processing parameters (gain G1 to G4, dynamic range Dr1 to Dr4, sensitivity time gain Gs1 to Gs4) in each series of image processing P1 to P4 may be stored beforehand in the storage device (not shown) or may be newly inputted by the operator.

Detailed description will be given to each series of image processing P1 to P4. In this example, the image processing unit 52 carries out the individual series of image processing P1 to P4 in parallel with respect to unit data X comprised of the frame data FD1 to FD100. In each image processing P1 to P4, series of image processing are carried out in parallel with different image processing parameters with respect to frame data FD of an identical time phase. More specific description will be given. To obtain ultrasonic images of the first frame, for example, the series of image processing P1 to P4 are carried out in parallel with respect to frame data FD1. Thereafter, to obtain ultrasonic images of the second frame, the series of image processing P1 to P4 are carried out in parallel with respect to frame data FD2. To obtain ultrasonic images of the third and following frames, the series of image processing P1 to P4 are sequentially carried out in parallel with respect to frame data FD3, FD4, . . . , FD100.

The digital scan converter 53 generates the respective ultrasonic images G1 to G4 with respect to each piece of frame data FD based on data obtained by the series of image processing P1 to P4 and causes the display unit 6 to display these images. The individual ultrasonic images G1 to G4 displayed on the display unit 6 are B-mode images different in image processing parameters with respect to frame data FD of an identical time phase. More specific description will be given. On the display unit 6, ultrasonic images G1 to G4 generated with different image processing parameters based on frame data FD1 are displayed as images of the first frame. In the next frame, or the second frame, ultrasonic images G1 to G4 generated with different image processing parameters based on frame data FD2 are displayed. Subsequently, ultrasonic images G1 to G4 are generated and displayed with respect to frame data FD3, FD4, . . . , FD100 in this order. On the display unit 4, therefore, four ultrasonic images G1 to G4 of an identical time phase generated with different image processing parameters are displayed as moving images corresponding to frame data FD1 to FD100.

With the ultrasonic diagnostic apparatus 1 in this example, the following can be implemented even after the end of a scan by the ultrasonic probe 2: it is possible to read echo data stored in the cine memory 51 and carry out image processing with different image processing parameters and to generate four ultrasonic images G1 to G4 and display the images in alignment. The echo data stored in the cine memory 51 at this time is data that has not undergone image processing at the image processing unit 52. Therefore, ultrasonic images G1 to G4 can be displayed by setting any image processing parameter to carry out image processing.

Description will be given to a modification to the above embodiment. In this modification, the image processing unit 52 carries out the respective series of image processing P1 to P4 in parallel with respect to the following data: divided unit data X1, X2, X3, X4 obtained by dividing unit data X comprised of the frame data FD1 to FD100 into four as illustrated in FIG. 5. In addition, the digital scan converter 53 generates the following ultrasonic images and displays the images: an ultrasonic image G1 corresponding to divided unit data X1; an ultrasonic image G2 corresponding to divided unit data X2; an ultrasonic image G3 corresponding to divided unit data X3; and an ultrasonic image G4 corresponding to divided unit data X4. Hereafter, concrete description will be given.

Each piece of divided data X1 to X4 is comprised of 25 pieces of frame data FD. Specifically, the divided unit data X1 is comprised of frame data FD1 to FD25; the divided unit data X2 is comprised of frame data FD26 to FD50; the divided unit data X3 is comprised of frame data FD51 to FD75; and the divided unit data X4 is comprised of frame data FD76 to FD100.

In each image processing P1 to P4 in this modification, series of image processing are carried out in parallel with different image processing parameters with respect to frame data FD of different time phases. Specifically, in the first image processing P1, image processing with respect to the divided unit data X1, that is, image processing with respect to frame data FD1 to FD25 is carried out; and in the second image processing P2, image processing with respect to the divided unit data X2, that is, image processing with respect to frame data FD26 to FD50 is carried out. In the third image processing P3, image processing with respect to the divided unit data X3, that is, image processing with respect to frame data FD51 to FD75 is carried out; and in the fourth image processing P4, image processing with respect to the divided unit data X4, that is, image processing with respect to frame data FD76 to FD100 is carried out.

To obtain ultrasonic images of the first frame, for example, the image processing unit 52 carries out the following series of processing in parallel: first image processing P1 with respect to frame data FD1; second image processing P2 with respect to frame data FD26; third image processing P3 with respect to frame data FD51; and fourth image processing P4 with respect to frame data FD76. Then it outputs data processed by the individual series of image processing P1 to P4 to the digital scan converter 53. To obtain ultrasonic images of the second frame, subsequently, the image processing unit 52 carries out the following series of processing in parallel: first image processing P1 with respect to frame data FD2; second image processing P2 with respect to frame data FD27; third image processing P3 with respect to frame data FD52; and fourth image processing P4 with respect to frame data FD77. Then it outputs data processed by the individual series of image processing P1 to P4 to the digital scan converter 53. For the third and following frames, the image processing unit 52 sequentially carries out the respective series of image processing P1 to P4 with respect to frame data FD of different time phases similarly with the foregoing. Then it outputs data obtained by the individual series of image processing P1 to P4 to the digital scan converter 53.

The ultrasonic images G1 to G4 displayed on the display unit 6 by the digital scan converter 53 are images generated based on frame data FD of different time phases. On the display unit 6, specifically, the following ultrasonic images are displayed as images of the first frame: ultrasonic image G1 generated based on frame data FD1; ultrasonic image G2 generated based on frame data FD26; ultrasonic image G3 generated based on frame data FD51; and ultrasonic image G4 generated based on frame data FD76. As images of the subsequent frame, or the second frame, the following images are displayed: ultrasonic image G1 generated based on frame data FD2; ultrasonic image G2 generated based on frame data FD27; ultrasonic image G3 generated based on frame data FD52; and ultrasonic image G4 generated based on frame data FD77. Also with respect to the third and following frames, ultrasonic images G1 to G4 sequentially generated based on frame data of different time phases are displayed similarly with the foregoing. On the display unit 4, therefore, four ultrasonic images G1 to G4 of different time phases generated with different image processing parameters are displayed as moving images.

Incidentally, the ultrasonic image G1 is a moving image corresponding to frame data FD1 to FD25; the ultrasonic image G2 is a moving image corresponding to frame data FD26 to FD50; the ultrasonic image G3 is a moving image corresponding to frame data FD51 to FD75; and the ultrasonic image G4 is a moving image corresponding to frame data FD76 to FD100.

Up to this point, description has been given to the invention based on the above embodiment. However, the invention can be variously modified without departing from the subject matter of the invention, needless to add. For example, the number of ultrasonic images displayed on the display unit 6 is not limited to four. The number of frames of the above unit frame data X is not limited to the above embodiment, either. Further, the number of divided pieces of the unit data X (the number of pieces of divided unit data) in the modification is not limited to four and it may be divided into a number corresponding to the number of displayed ultrasonic images.

The ultrasonic images are not limited only to B-mode images. For example, M-mode images may be displayed together with B-mode images. Also in this case, image processing is carried out with different image processing parameters with respect to echo data stored in the cine memory 51 and multiple M-mode images are displayed. In addition, a color Doppler image may be displayed so that it is overlaid on a B-mode image. In this case, signals obtained by the transmission and reception of an ultrasonic wave are subjected to Doppler processing and the velocity, variance, and power of the echo source are stored in the cine memory 51. These pieces of data are read and image processing is carried out with different image processing parameters to generate multiple color Doppler images. Then these color Doppler images are overlaid on multiple B-mode images.

In the modification, in addition, the following measure may be taken: with respect to the divided unit data X1 to X4, image processing is carried out with different image processing parameters as mentioned above and multiple ultrasonic images are generated and displayed; and further with respect to at least any one piece of divided unit data, image processing is carried out with different image processing parameters and multiple ultrasonic images are generated and displayed. For example, with respect to the divided unit data X1, image processing is carried out with image processing parameters having different numeric values to generate and display multiple ultrasonic images; and further, with respect to the divided unit data X2 to X4, image processing is respectively carried out with predetermined image processing parameters. Then one ultrasonic image corresponding to the divided unit data X2, one ultrasonic image corresponding to the divided unit data X3, and one ultrasonic image corresponding to the divided unit data X4 are displayed. In this case, the parameters for image processing with respect to the divided unit data X2 to X4 are made different from one another.

Many widely different embodiments of the invention may be configured without departing from the spirit and the scope of the present invention. It should be understood that the present invention is not limited to the specific embodiments described in the specification, except as defined in the appended claims.

1. An ultrasonic diagnostic apparatus comprising:
a storage unit configured to store echo data obtained by transmitting and receiving an ultrasonic wave to and from a subject;
an image processing unit configured to perform image processing with different image processing parameters with respect to the echo data stored in the storage unit; and
a display processing unit configured to generate a plurality of ultrasonic images based on the processed echo data to display the plurality of ultrasonic images.

2. The ultrasonic diagnostic apparatus according to claim 1, wherein:
the ultrasonic images are either moving images or still images.

3. The ultrasonic diagnostic apparatus according to claim 1, wherein:
with respect to frame data of an identical time phase comprised of the echo data obtained by transmitting and receiving the ultrasonic wave to and from the subject, the image processing unit is configured to perform image processing with the different image processing parameters and the display processing unit is configured to generate and display the plurality of ultrasonic images.

4. The ultrasonic diagnostic apparatus according to claim 2, wherein:
with respect to frame data of an identical time phase comprised of the echo data obtained by transmitting and receiving the ultrasonic wave to and from the subject, the image processing unit is configured to perform image processing with the different image processing parameters and the display processing unit is configured to generate and display the plurality of ultrasonic images.

5. The ultrasonic diagnostic apparatus according to claim 3, wherein:
with respect to unit data comprised of a plurality of pieces of the frame data, the image processing unit is configured to perform image processing on each piece of the frame data with the different image processing parameters and the display processing unit is configured to generate the plurality of ultrasonic images with respect to each piece of frame data to display moving images.

6. The ultrasonic diagnostic apparatus according to claim 4, wherein:
with respect to unit data comprised of a plurality of pieces of the frame data, the image processing unit is configured to perform image processing on each piece of the frame data with the different image processing parameters and the display processing unit is configured to generate the plurality of ultrasonic images with respect to each piece of frame data to display moving images.

7. The ultrasonic diagnostic apparatus according to claim 1, wherein:
with respect to frame data of different time phases comprised of the echo data obtained by transmitting and receiving the ultrasonic wave to and from the subject, the image processing unit is configured to perform image processing with the different image processing parameters and the display processing unit is configured to generate and display the plurality of ultrasonic images.

8. The ultrasonic diagnostic apparatus according to claim 2, wherein:
with respect to frame data of different time phases comprised of the echo data obtained by transmitting and receiving the ultrasonic wave to and from the subject, the image processing unit is configured to perform image processing with the different image processing parameters and the display processing unit is configured to generate and display the plurality of ultrasonic images.

9. The ultrasonic diagnostic apparatus according to claim 7, wherein:
with respect to each piece of divided unit data obtained by dividing the unit data comprised of a plurality of pieces of the frame data into a predetermined number of frames, the image processing unit is configured to perform image processing on each piece of frame data with the different image processing parameters and the display processing unit is configured to generate the plurality of ultrasonic images with respect to each piece of frame data to display moving images.

10. The ultrasonic diagnostic apparatus according to claim 8, wherein:
with respect to each piece of divided unit data obtained by dividing the unit data comprised of a plurality of pieces of the frame data into a predetermined number of frames, the image processing unit is configured to perform image processing on each piece of frame data with the different image processing parameters and the display processing unit is configured to generate the plurality of ultrasonic images with respect to each piece of frame data to display moving images.

11. The ultrasonic diagnostic apparatus according to claim 9, wherein:
with respect to at least one piece of the divided unit data, the image processing unit is configured to perform image processing with the different image processing parameters and the display processing unit is configured to generate and display the plurality of ultrasonic images.

12. The ultrasonic diagnostic apparatus according to claim 10, wherein:
with respect to at least one piece of the divided unit data, the image processing unit is configured to perform image processing with the different image processing parameters and the display processing unit is configured to generate and display the plurality of ultrasonic images.

13. The ultrasonic diagnostic apparatus according to claim 1, wherein:
the image processing unit is configured to perform image processing with the different image processing parameters in parallel.

14. The ultrasonic diagnostic apparatus according to claim 2, wherein:
the image processing unit is configured to perform image processing with the different image processing parameters in parallel.

15. The ultrasonic diagnostic apparatus according to claim 3, wherein:
the image processing unit is configured to perform image processing with the different image processing parameters in parallel.

16. The ultrasonic diagnostic apparatus according to claim 7, wherein:
the image processing unit is configured to perform image processing with the different image processing parameters in parallel.

17. The ultrasonic diagnostic apparatus according to claim 9, wherein:
the image processing unit is configured to perform image processing with the different image processing parameters in parallel.

18. The ultrasonic diagnostic apparatus according to claim 1, wherein:
the image processing parameters are gain, dynamic range, and sensitivity time gain.

19. A method of displaying ultrasonic images comprising the steps of:
storing echo data obtained by transmitting and receiving an ultrasonic wave to and from a subject;
performing image processing with different image processing parameters with respect to the echo data stored in the storage unit;
generating a plurality of ultrasonic images based on the echo data that underwent image processing at the image processing unit; and
displaying the plurality of ultrasonic images.

20. An ultrasonic image display control program causing a computer to carry out:
an image processing function that causes the computer to read echo data obtained by transmitting and receiving an ultrasonic wave to and from a subject, from a storage unit and to perform image processing on the echo data with different image processing parameters; and
a display processing function that causes the computer to generate a plurality of ultrasonic images based on the echo data that underwent image processing by the image processing function and to display the plurality of ultrasonic images in alignment.
